(19) United States
(12) Patent Application Publication
(10) Pub. No.: US 2006/0293312 A1
(43) Pub. Date: Dec. 28, 2006

(54) Method of improved diuresis in individuals with impaired renal function

(76) Inventors: Howard Dittrich, San Diego, CA (US); Brian Farmer, San Diego, CA (US); Randy Woods, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/454,665

(22) Filed: Jun. 16, 2006

Related U.S. Application Data
(63) Continuation-in-part of application No. 10/830,617, filed on Apr. 23, 2004.
(60) Provisional application No. 60/466,007, filed on Apr. 25, 2003.

(51) Int. Cl. A61K 31/549; A61K 31/522; A61K 31/4965

(52) U.S. Cl. 514/223.5; 514/263.34; 514/263.32; 514/255.05

(57) ABSTRACT

Disclosed is a pharmaceutical composition comprising a therapeutically effective amount of KW-3902, or a salt, ester, amide, metabolite, or prodrug thereof, and a non-adenosine modifying diuretic. Also disclosed are methods of inducing a diuretic effect in an animal comprising the step of administering a therapeutically effective amount of KW-3902, or a salt, ester, amide, metabolite, or prodrug thereof, in combination with second pharmaceutical composition capable of inducing a diuretic effect.

### RELATED APPLICATIONS

The present application is a continuation-in part of U.S. Ser. No. 10/830,617, filed on Apr. 23, 2004, by Widder, et al., and entitled “METHOD OF TREATING PATIENTS REFRACTORY TO STANDARD DIURETIC THERAPY,” which in turn claims priority to the Provisional Application Ser. No. 60/466,007, filed on Apr. 25, 2003, by Kenneth J. Widder, and entitled “METHOD OF TREATING PATIENTS REFRACTORY TO STANDARD DIURETIC THERAPY,” both of which are incorporated herein by reference in their entirety.

### FIELD OF THE INVENTION

The present invention relates to methods of improving renal function in individuals with congestive heart failure comprising administering to an individual in need thereof a low-dose of an adenosine Al receptor antagonist.

### SUMMARY OF THE INVENTION

Provided herein are methods of treating an individual with impaired renal function. In some embodiments, the methods include the steps of identifying an individual with impaired renal function and administering to the individual a pharmaceutical composition having less than about 10 mg KW-3902, or a pharmaceutically acceptable salt, ester, amide, metabolite, or prodrug thereof.

In some embodiments, the identification step can include the step of identifying an individual with a creatinine clearance rate of less than about 80 mg/dL to about 20 mg/dL.

In some embodiments, the individual suffers from congestive heart failure. In some embodiments, the individual is not refractory to standard diuretic therapy, whereas in other embodiments, the individual is refractory to standard diuretic therapy.

In some embodiments, the pharmaceutical composition includes about 1.5 to about 5 mg KW-3902. Accordingly, in some embodiments, the pharmaceutical composition includes about 2.0 mg to about 3.0 mg KW-3902. Preferably, the pharmaceutical composition includes about 2.5 mg KW-3902.

Some embodiments of the methods provided herein also include the step of administering to the individual a therapeutically effective amount of a non-adenosine modifying diuretic. For example, in some embodiments, a therapeutically effective amount of a proximal diuretic, a loop diuretic, or a distal diuretic is administered to the individual. Accordingly, in some embodiments, a therapeutically effective amount of a non-adenosine modifying diuretic selected from hydrochlorothiazides, furosemide, torsemide, bumetanide, ethacrynic acid, piretanide, spironolactone, triamterene, and amiloridehiazides is administered to the individual. Preferably, the non-adenosine modifying diuretic is fursoemide.

In some embodiments, KW-3902 can be administered substantially simultaneously with a non-adenosine modifying diuretic. In other embodiments, KW-3902 and the non-adenosine modifying diuretic are administered sequentially.

### BREIF DESCRIPTION OF THE DRAWINGS

FIG. 1 shows the urine volume following treatment with placebo or with KW-3902 in individuals with acute fluid overload.

FIG. 2 shows the total dose of IV furosemide following treatment with placebo or with KW-3902 in individuals with acute fluid overload.

FIG. 3 shows the change in urine volume over baseline following treatment with placebo or with KW-3902 in individuals refractory to diuretic therapy.

FIG. 4 shows the change in creatinine clearance over baseline following treatment with placebo or with KW-3902 in individuals refractory to diuretic therapy.

### DETAILED DESCRIPTION OF THE PREFERRED EMBODIMENT

Aspects provided herein are directed to methods of improving renal function using a therapeutically effective amount of KW-3902 less than about 10 mg. Other aspects provided herein are directed to methods of treating patients using a therapeutically effective amount of KW-3902, or a salt, ester, amide, metabolite, or prodrug thereof, wherein the amount of KW-3902 is less than about 10 mg and a non-adenosine modifying diuretic. Yet other aspects are directed to methods of improving diuresis while maintaining renal function in individuals with fluid overload using a therapeutically effective amount of KW-3902, or a salt, ester, amide, metabolite, or prodrug thereof, and a non-adenosine modifying diuretic.

The term “therapeutically effective amount” as used herein refers to that amount of a composition being administered which will relieve to some extent one or more of the signs or symptoms of the disorder being treated, or an amount effective to achieve the desired effect.

In certain embodiments, the individual being treated by the methods of the present invention suffers from renal impairment. In other embodiments, the individual does not suffer from renal impairment. For example, these individuals can exhibit a urine creatinine clearance rate of about 20 mg/dL to about 80 mg/dL. These individuals include those who suffer from heart failure, such as congestive heart failure, or other maladies that result in fluid overload, without having yet disrupted normal kidney function. In some embodiments, the individual being treated by the methods of the present invention is refractory to standard diuretic therapy. In other embodiments, the individual is not refractory to standard diuretic therapy.

Other aspects are related to methods of preventing the deterioration of renal function in individuals comprising administering a therapeutically effective amount of KW-3902, or a salt, ester, amide, metabolite, or prodrug thereof, wherein the therapeutically effective amount is less than about 10 mg. In some embodiments, a non-adenosine modifying diuretic is also administered.

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

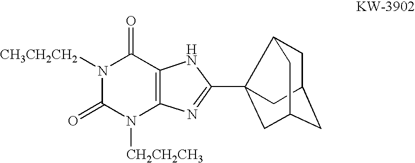

KW-3902 and related compounds useful in the practice of the present invention are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

A number of non-adenosine modifying diuretics are known in the art. Their examples include hydrochlorothiazides, furosemide, torsemide, bumetanide, ethacrynic acid, piretanide, norsemide, spironolactone, triamterene, metolazone and amiloridethiazides.

A significant problem encountered in treating certain conditions with individual medications is that following a course of therapy the patients become refractory to the treatment, and begin to respond less and less to the medication until they do not respond at all. This problem is very common in patients who suffer from, for example, congestive heart failure, and are treated with diuretics.

Individual diuretics act on a specific segment of nephrons, e.g., proximal tubule, loop of Henle, or distal tubule. One mechanism by which diuretics increase urine volume is that they inhibit reabsorption, of sodium and accompanying water passing through the nephron. Thus, for example, a loop diuretic inhibits reabsorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium reabsorption elsewhere in the nephron; the other is to feedback via adenosine Al receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

The present inventors have made the surprising discovery that AA₁RAs (e,g., KW-3902) have beneficial effects on kidney function that are not dose-dependent. Unexpectedly, the beneficial effects of AA₁RAs (e.g., KW-3902) on kidney function are seen at lower doses of AA₁RAs (e.g., less than about 10 mg KW-3902) than are necessary to achieve the maximum beneficial effects on diuresis. Accordingly, methods described herein directed to treatment regiments for the improvement and/or maintenance of renal function included administration of AA₁RAs (e.g., KW-3902) at a dose lower than the amount required to achieve maximal diuretic effects. In accordance with the unexpectedly large beneficial effect on kidney function at lower doses of AA₁RAs, embodiments of the methods provided herein include the step of administering less than or equal to about about 10 mg, for example at least about 5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, 10 mg, KW-3902.

The present inventors have also discovered that the combination of KW-3902 with a standard diuretic is beneficial to patients who are refractory to standard therapy. KW-3902 also blocks the TGF mechanism mediated by adenosine (via A₁ receptors) described above. This ultimately allows for increased GFR and improved renal function, which ultimately results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, which can counteract the adverse effect of some diuretics, such as proximal diuretics, which activate or promote TGF.

The combinations described herein act synergistically to further improve renal function for continued diuresis. In addition, most CHF patients are also on additional diuretics. The combination allows for greater efficacy of other more distally acting diuretics by improving renal blood flow, renal function, and in some cases, drug delivery.

Thus, in one aspect, the present invention is related to a pharmaceutical composition comprising a therapeutically effective amount of KW-3902, or a pharmaceutically acceptable salt, ester, amide, metabolite, or prodrug thereof, and a non-adenosine modifying diuretic. In some embodiments, the pharmaceutical composition contains less than about 10 mg KW-3902, or a pharmaceutically acceptable salt, ester, amide, metabolite, or prodrug thereof.

In some embodiments, the non-adenosine modifying diuretic is a proximal diuretic, i.e., a diuretic that principally acts on the proximal tubule. Examples of proximal diuretics include, but are not limited to, acetazolamide, methazolamide, and dichlorphenamide. Carbonic anhydrase inhibitors are known to be diuretics that act on the proximal tubule, and are therefore, proximal diuretics. Thus, in certain embodiments, the present invention relates to the combination of KW-3902 with a carbonic anhydrase inhibitor. Combinations of KW-3902 with any proximal diuretic now known or later discovered are within the scope of the present invention. In some embodiments, the pharmaceutical composition includes a proximal diuretic and less than about 10 mg KW-3902, or a pharmaceutically acceptable salt, ester, amide, metabolite, or prodrug thereof.

In other embodiments, the non-adenosine modifying diuretic is a loop diuretic, i.e., a diuretic that principally acts on the loop of Henle. Examples of loop diuretics include, but are not limited to, furosemide (LASIX®), bumetanide (BUMEX®), and torsemide (TOREM®). Combinations of KW-3902 with any loop diuretic now known or later discovered are within the scope of the present invention. In some embodiments, the pharmaceutical composition includes a loop diuretic and less than about 10 mg KW-3902, or a pharmaceutically acceptable salt, ester, amide, metabolite, or prodrug thereof.

In yet other embodiments, the non-adenosine modifying diuretic is a distal diuretic, i.e., a diuretic that principally acts on the distal nephron. Examples of distal diuretics include, but are not limited to, metolazone, thiazides and amiloride. Combinations of KW-3902 with any distal diuretic now known or later discovered are within the scope of the present invention. In some embodiments, the pharmaceutical composition includes a distal diuretic and less than about 10 mg KW-3902, or a pharmaceutically acceptable salt, ester, amide, metabolite, or prodrug thereof.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which KW-3902 is converted within the cells of a mammal. The pharmaceutical compositions of the present invention may include a metabolite of KW-3902 instead of KW-3902. The scope of the methods of the present invention includes those instances where KW-3902 is administered to the patient, yet the metabolite is the bioactive entity.

Some of the metabolites of KW-3902 are known. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), 8-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo [3.3.1.0^{3,7}]nonyl)-3-propylxanthine.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In another aspect, the present invention relates to a method of treating an individual with impaired renal function, comprising identifying a patient in need thereof, and administering to the patient a therapeutically effective amount of an AA₁RA, (e.g., KW-3902), wherein the therapeutically effective amount is less than or equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, 10 mg.

Renal function refers to the ability the kidney to excrete waste and maintain a proper chemical balance. Typically, renal function is measured by plasma concentrations of creatinine, urea, and electrolytes to determine renal function. Creatinine is a byproduct of normal muscle metabolism that is produced at a fairly constant rate in the body and normally filtered by the kidneys and excreted in the urine. It will be appreciated that any method known to those skilled in the art for measuring renal function can be used in the methods described herein. For example, serum creatinine levels, urine creatinine levels, and glomerular filtration rate (GFR) can be used to assess renal function.

In some embodiments, impaired renal function refers to a GFR of less than about 80 mL/min, for example about 20 mL/min, 30 mL/min, 40 mL/min, 50 mL/min, 60 mL/min 70 mL/min or 75 mL/min, or any number in between prior to hospitalization. Accordingly, in some embodiments, the patient exhibits mildly impaired renal function (e.g., a GFR of about 50 to about 80 mL/min). In some embodiments, the patient exhibits moderately impaired renal function (e.g., a GFR of about 30 mL/min to about 50 mL/min). In yet other embodiments, the patient exhibits severely impaired renal function (e.g., a GFR of equal to or less than about 30 mL/min).

Optionally, in some embodiments, the methods include the step of administering a non-adenosine modifying diuretic to said individual.

In another aspect, the present invention relates to a method of inducing a diuretic effect in an animal comprising identifying a patient in need thereof and administering to the patient a therapeutically effective amount of KW-3902, or a pharmaceutically acceptable salt, ester, amide, metabolite, or prodrug thereof, in combination with a second pharmaceutical composition capable of inducing a diuretic effect. In some embodiments, the animal is refractory to standard diuretic therapy.

The methods of the present invention are effective in a patient. The patient may be an animal. The animal may be a mammal. The mammal may be selected from the group consisting of mice, rats, rabbits, guinea pigs, dogs, cats, sheep, goats, cows, primates, such as monkeys, chimpanzees, and apes, and humans. In some preferred embodiments, the animal is a human.

In some embodiments, the administering step comprises administering KW-3902 and the diuretic nearly simultaneously. These embodiments include those in which the two compounds are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc. In some embodiments, a patient is infused with an intravenous formulation of one compound prior to the infusion of an intravenous formulation of the other compound. In these embodiments, the infusion may take some time, such as a few minutes, a half hour, or an hour, or longer. If the two intravenous infusions are done one right after the other, such administration is considered to be nearly simultaneously within the scope of the present disclosure, even though there was a lapse of some time between the start of one infusion and the start of the next infusion.

In other embodiments the administering step comprises administering one of KW-3902 and the diuretic first and then administering the other one of KW-3902 and the diuretic. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally. In further embodiments, the patient may receive both compounds on a routine or continuous basis, such a continuous infusion of the compound through an IV line.

In some embodiments, KW-3902 is administered in a dose of at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, 10 mg, 15 mg, 30 mg, 60 mg, or 100 mg, or higher. In some embodiments, the administered KW-3902 is in an injectable form, while in other embodiments, the administered KW-3902 is in a solid formulation.

In another aspect, the present invention relates to a method of maintaining or restoring the diuretic effect of a non-adenosine modifying diuretic in a patient comprising identifying a patient in need thereof, and administering to the patient a therapeutically effective amount of KW-3902, or a pharmaceutically acceptable salt, ester, amide, metabolite, or prodrug thereof, in combination with said non-adenosine modifying diuretic. In some embodiments, the amount of KW-3902 is less than ore equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, 10 mg. Optionally, a non-adenosine modifying diuretic is also administered to the individual.

In certain embodiments, the diuretic used in the methods of the present invention is furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenous, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

In yet another aspect, the present invention relates to a method of maintaining or restoring renal function in a patient comprising identifying a patient in need thereof, and administering a therapeutically effective amount of KW-3902, or a pharmaceutically acceptable salt, ester, amide, metabolite, or prodrug thereof, wherein said therapeutically effective amount is less than or equal to about 10 mg, 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, 10 mg. Optionally, the KW-3902 is administered with a second pharmaceutical composition capable of inducing a diuretic effect.

In yet another aspect, the present invention relates to a method of maintaining or restoring renal function in a patient comprising identifying a patient in need thereof, and administering a therapeutically effective amount of KW-3902, or a pharmaceutically acceptable salt, ester-amide, metabolite, or prodrug thereof, wherein the therapeutically effective amount of KW-3902 or pharmaceutically acceptable salt, ester, amide, metabolite, or prodrug thereof is less than or equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, or 10 mg. Optionally, the KW-3902 or pharmaceutically acceptable salt, ester, amide, metabolite, or prodrug thereof is administered in combination with second pharmaceutical composition capable of inducing a diuretic effect.

In the context of the present disclosure, by “maintaining” renal function it is meant that the renal function, as measured by creatinine clearance rate, remains unchanged for a period of time after the start of the therapy. In other words, by “maintaining” renal function it is meant that the rate of renal impairment, i.e., the rate of decrease in the urine creatinine clearance rate, is slowed or arrested for a period of time, however brief that period may be. By “restoring” renal function it is meant that the renal function, as measured by urine creatinine clearance rate, has improved, i.e., has become higher, after the start of the therapy.

In certain embodiments, the second pharmaceutical composition comprises a loop diuretic and a distal diuretic.

In a further aspect, the present invention relates to a method of treating a patient with a pharmaceutical composition as described herein. In some embodiments, the patient is refractory to standard diuretic therapy.

Certain patients who suffer from a cardiac condition, such as congestive heart failure, later develop renal impairment. The present inventors have discovered that if a patient presented with a cardiac condition, and little to no renal impairment, is treated with a pharmaceutical composition as described herein, the onset of renal impairment is delayed or arrested, compared to a patient who receives standard treatment. Thus, aspects of the present invention relate to a method of preventing the deterioration of renal function, delaying the onset of renal impairment, or arresting the progress of renal impairment in a patient comprising identifying a patient in need thereof, and administering a therapeutically effective amount of KW-3902, or a salt, ester, amide, metabolite, or prodrug thereof, wherein the therapeutically effective amount of KW-3902 or pharmaceutically acceptable salt, ester, amide, metabolite, or prodrug thereof is less than or equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, or 10 mg. Optionally, the KW-3902 or pharmaceutically acceptable salt, ester, amide, metabolite, or prodrug thereof is administered in combination with second pharmaceutical composition capable of inducing a diuretic effect, such as a non-adenosine modifying diuretic.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved. Thus, in the context of the present invention, increasing the urine output volume, decreasing the level of serum creatinine, or increasing creatinine clearance, may be considered treatment, even if the patient is not cured or does not generally feel better.

In another aspect, the present invention relates to a method of treating a patient suffering from CHF comprising identifying a patient in need thereof, and administering to said patient a therapeutically effective amount of KW-3902, or a pharmaceutically acceptable salt, ester, amide, metabolite, or prodrug thereof, in combination with second pharmaceutical composition, wherein the therapeutically effective amount of KW-3902 or pharmaceutically acceptable salt, ester, amide, metabolite, or prodrug thereof is less than or equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, or 10 mg. Optionally, the KW-3902 or pharmaceutically acceptable salt, ester, amide, metabolite, or prodrug thereof is administered in combination with second pharmaceutical composition capable of inducing a diuretic effect. 10055] In a further aspect, the present invention relates to a method of improving overall health outcomes, decreasing morbidity rates, or decreasing mortality rates in patients comprising identifying a patient in need thereof, and administering to said patient a therapeutically effective amount of KW-3902, or a pharmaceutically acceptable salt, ester, amide, metabolite, or prodrug thereof, wherein the therapeutically effective amount of KW-3902 or pharmaceutically acceptable salt, ester, amide, metabolite, or prodrug thereof is less than or equal to about 10 mg, e.g., at least about 0.5 mg, 1.0 mg, 1.5 mg, 2.0 mg, 2.5 mg, 3.0 mg, 3.5 mg, 4.0 mg, 4.5 mg, 5 mg, 6 mg, 7 mg, 8 mg, 9 mg, or 10 mg. Optionally, the KW-3902 or pharmaceutically acceptable salt, ester, amide, metabolite, or prodrug thereof is administered in combination with second pharmaceutical composition capable of inducing a diuretic effect.

Overall health outcomes are determined by various means in the art. For example, improvements in morbidity and/or mortality rates, improvements in the patient's general feelings, improvements in the quality of life, improvements in the level of comfort at the end of life, and the like, are considered when overall health outcome are determined. Mortality rate is the number of patients who die while undergoing a particular treatment for a period of time compared to the overall number of patients undergoing the same or similar treatment over the same period of time. Morbidity rates are determined using various criteria, such as the frequency of hospital stays, the length of hospital stays, the frequency of visits to the doctor's office, the dosage of the medication being administered, and the like.

In some embodiments, the patient whose overall health outcome, morbidity and/or mortality rate is being improved suffers from CHF. In other embodiments, the patient suffers from renal impairment.

Other AA₁RAs are known in the art, for example, BG 9719, described in U.S. Patent Application Publication No. 2002/0115687 A1. BG 9719 is also a xanthine-derivative compound, whose structure bears some similarity to that of KW-3902. However, the present inventors have surprisingly discovered that despite the structural similarity of these compounds, they behave remarkably differently in various ways. For example, solid formulations of KW-3902 are easily obtained, and in fact disclosed in U.S. Pat. No. 6,254,889. These formulations are found to be very stable at room temperature for over 3 years. Pharmacokinetic data from single administration in normal volunteers of both IV and oral dosage forms of KW-3902 show a bioavailability of 25% for the oral dosage form and demonstrate its diuretic effect. The 20 mg oral dose group showed a 2× increase in urine volume compared to the placebo group at over the first two hours after treatment. (Unpublished clinical study data.) By contrast, literature references show that BG 9719 cannot be formulated in a solid form. For example, it has been reported that the development program for BG 9719 has “been hampered by its poor solubility and stability as well as the lack of a suitable oral formulation” (B. Ticho et al, Drug Development Research 58:486-492 (2003)).

The two compounds also show significantly different affinity for adenosine A₁ receptors and are not equally selective for adenosine A₁ receptors over adenosine A₂ₐ receptors. The data for the two compounds are shown in the table below.

| Compound | A₁K_{I} (nM) | A₂ₐK_{I} (nM) |
|---|---|---|
| BG 9719 | 0.45 ± .04 | 1100 ± 318 |
| KW 3902 | 0.72 ± .12 | 108 ± 15 |

These data show BG 9719 to have a 60% great affinity for the Al receptor than KW-3902. Selectivity for the A₁ receptor over the A₂a receptor by BG 9719 is 16 times greater than selectivity by KW-3902. The data for BG 9719 were obtained from the U.S. Patent Application Publication No. 2002/0115687 A1, while the data for KW-3902 were published in Pfister, J. R, et al, J. Med. Chem. 1997, 40, 1773-1778.

Furthermore, at equi-potent dosages of KW-3902 and BG 9719 (unpublished clinical study data; Wolff et al, Drug Development Research 45:166-177 (1998)), when compared to placebo, KW-3902 shows significantly greater diuretic effect than BG 9719. For the purposes of the analysis below, the equi-potent dose of KW 3902 was calculated by multiplying the ratio of the A₁K₁ by the dose of BG 9719 i.e. (0.72/0.45)×0.3 mg/kg=0.48 mg/kg.

| | | | % Urine volume increase |
|---|---|---|---|
| Compound | A₁K_{I} (nM) | Equi-potent Dose | over placebo, 3 hr |
| BG 9719 | 0.45 ± .04 | .3 mg/kg or 20 mg | 33% |
| KW-3902 | 0.72 ± .12 | .48 mg/kg or 30 mg | 195% |

These data collectively show that while BG 9719 and KW-3902 have some structural similarity, their pharmacokinetic activity and physiological function is quite surprisingly different, with KW-3902 providing an unexpected advantage over BG 9719.

Methods of the present invention described herein may be practiced by using, instead of KW-3902, a xanthine-derivative compound of Formula I or a pharmaceutically acceptable salt thereof, 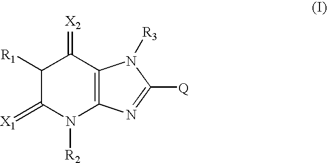
where
each of X₁ and X₂ independently represents oxygen or sulfur;
Q represents: 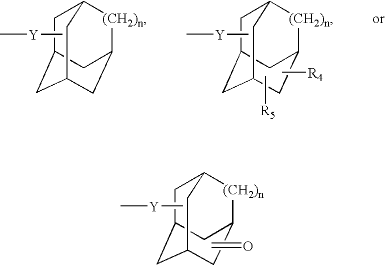
   where Y represents a single bond or alkylene having 1 to 4 carbon atoms, n represents 0 or 1;

each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or

R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 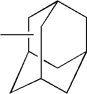
then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represents hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R₁ is propyl, 2-hydroxypropyl, 2-oxopropyl or 3-oxopropyl; R₂ is propyl, 2-hydroxypropyl or 3-hydroxypropyl.

In some embodiments Q is 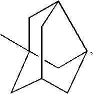
while in other embodiments Q is

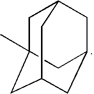

In other embodiments, Q is 9-hydroxy, 9-oxo or 6-hydroxy substituted 3tricyclo[3.3.1.0^{3,7}]nonyl, or 3-hydroxy-1tricyclo[3.3.1.1^{3,7}]decyl.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvilnylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the presented invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservated. The compositions may take such forms as suspensions, solutions or emulsions in only or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpqlar surfactant Polysorbate 8OTM , and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, which is incorporated by reference herein in its entirety, including any drawings.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.01 to 1000 mg/kg of the patient's body weight. The dosage may be a single one or a series of two or more given in the course of one or more days, as is needed by the patient.

The daily dosage regimen of KW-3902 for an adult human patient may be, for example, an oral dose of between 0.1 mg and 500 mg, preferably between I mg and 250 mg, e.g. 5 to 200 mg or an intravenous, subcutaneous, or intramuscular dose of between 0.01 mg and 500 mg, preferably between 0.1 mg and 200 mg, e.g. 1 to 100 mg of the pharmaceutical compositions of the present invention or a pharmaceutically acceptable salt thereof calculated as the free base, the composition being administered I to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

In certain embodiments, KW-3902 is administered in conjunction with a diuretic. In these aspects, the dose of the diuretic is that which constitutes standard diuretic therapy. Those of skill in the art know what dosage of diuretics to administer to a patient in need thereof. However, because of the diuretic effect of KW-3902, the need for higher doses of the diuretic are eliminated when KW-3902 is administered to a patient in conjunction with the diuretic.

Dosage amount and interval may be adjusted individually to provide plasma levels of the active moiety that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

Where a range of values is provided, it is understood that each intervening value, to the tenth of the unit of the lower limit unless the context clearly dictates otherwise, between the upper and lower limit of that range and any other stated or intervening value in that state range, is encompassed within the invention. The upper and lower limits of these smaller rangers may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

### Example 1

### Treatment of Individuals with Fluid Overload and Renal Impairment

A double-blind, randomized multi-center, placebo controlled study was conducted as follows: Approximately 157 subjects were randomized to yield 144 evaluable subjects in an intent-to treat analysis conducted at approximately 50 sites. The study population included males and females at least 18 years of age with New York Heart Association Class II-IV CHF. All subjects had an estimated creatinine clearance between 20 mL/min and 80 mL/min. The average serum creatinine for all individuals at entry was 1.75 mg/dL. All subjects were taking an oral loop diuretic. The demographic data for the study is presented in Table 1 below.

TABLE 1
STUDY DEMOGRAPHICS

| | KW-3902 | | | | |
|---|---|---|---|---|---|
| | Placebo | 2.5 mg | 15 mg | 30 mg | 60 mg |
| n = (ITT population) | 27 | 29 | 30 | 29 | 29 |
| Age (mean yrs) | 67 | 64 | 69 | 66 | 67 |
| Sex (% M/% F) | 74/26 | 66/34 | 65/35 | 70/30 | 69/31 |
| NYHA Class II (%) | 4 | 0 | 0 | 3 | 7 |
| NYHA Class III (%) | 52 | 41 | 58 | 47 | 52 |
| NYHA Class IV (%) | 44 | 59 | 42 | 50 | 41 |

Study visits included pre-treatment days −2 to −1, days 1 to 3 of the Treatment Period, day 4/early Termination and a follow up contact at day 30. Procedures and observations included medical history, physical examination, classification of CHF, vital signs, body weight, CHF signs and symptom scores, Holter monitor recording, chest X-ray, CBC chemistries, creatinine clearance, fluid intake, and urine output.

On treatment days, individuals received KW-3902 intravenously over 120 minutes at one of four doses (2.5 mg, 15 mg, 30 mg, or 60 mg) vs. placebo as both monotherapy and concomitant therapy with diuretics. KW-3902 (or placebo) was administered on days 1 through 3. On day 1, KW-3902 (or placebo) was administered as a monotherapy. 6 hours after administration of KW-3902, IV loop diuretic was given to all treatment groups as needed. On days 2 and 3 KW-3902 was administered as combination therapy with intravenous furosemide, if clinically indicated. Final laboratory data aware collected on day 4 or early termination. A follow-up phone contact was conducted on day 30.

As shown in Table 2 below, individuals receiving as low as 2.5 mg KW-3902 exhibited an improvement in kidney function as measured by serum creatinine levels compared to the baseline levels. This effect was 7 times the effect seen in individuals who had received placebo. Further, as demonstrated by the changes in serum creatinine levels in individuals receiving the highest dose of KW-3902 (i.e., 60 mg), this effect was surprisingly not dose-dependent.

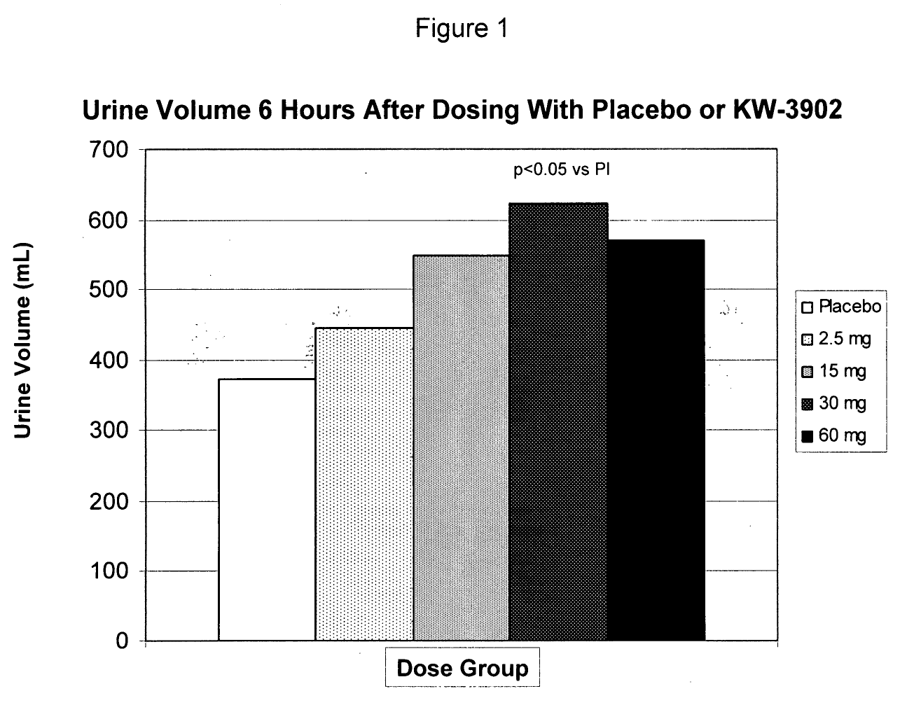

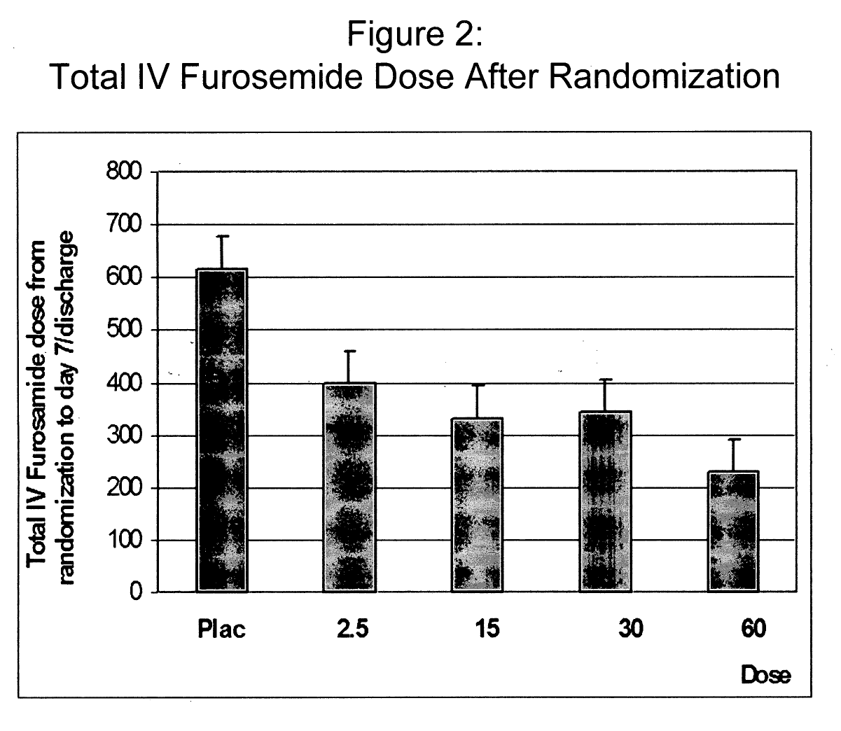

The combination of KW-3902 and furosemide had a synergistic beneficial effect on diuresis, as measured by urine output. In contrast to the effects on kidney function, the diuretic effects were dose-dependent at 2.5 mg, 15 mg, and 30 mg, as shown in Table 1 and FIG. 1. Individuals receiving KW-3902 also required less furosemide, as demonstrated in Table 1 and FIG. 2.

TABLE 2

| | | | | | | Change in | |
|---|---|---|---|---|---|---|---|
| | | | | | Change in | creatinine | |
| | | | | | creatinine | baseline to | |
| | | % Early Termination | Total IV | 6 hr. urine | baseline | last final | Cr increase ≧0.3 |
| Dose | n = | due to adequate | furosemide | volume, day 1 | to day 3 | observation | mg/dL any time |
| Group | (ITT pop) | diuresis | dose (mg) | (mL) | (mg/dL) | (mg/dL) | during hosp. (%) |
| Placebo | 27 | 4 | 606 | 374 | 0.00 | −0.01 | 18.5 |
| 2.5 mg | 29 | 14 | 397 | 445 | −0.08 | −0.07 | 10.3 |
| 15 mg | 31 | 39 | 331* | 531 | −0.05 | −0.04 | 9.7 |
| 30 mg | 30 | 30 | 342 | 631* | −0.11 | −0.09 | 10.3 |
| 60 mg | 29 | 38 | 229* | 570 | +.01 | 0.03 | 20.7 |

ITT pop = intent to treat population
Cr = serum creatinine levels
*p < 0.05

### Example 2

### Treatment of Individuals with Fluid Overload and Renal Impairment

A patient with fluid overload, as manifested by peripheral edema, dyspnea, and/or other signs or symptoms presents to the hospital, clinic, or doctor's office. The patient also shows some degree of renal impairment. In addition to standard of care therapy which would include IV diuretics, e.g., IV furosemide, bumetanide and/or oral metolazone, the patient is also given 2.5 mg of KW-3902 in injectable form. The patient is administered 2.5 mg of KW-3902 and 40 mg of furosemide at 24 hour intervals or more frequently as needed. The patient's fluid intake and output, urine volume, serum and urine creatinine levels, electrolytes and cardiac function are monitored.

At the discretion of the attending physician, the dosage of KW-3902 can be increased to 15 mg, 30 mg, or 60 mg either during the treatment or as the initial dose. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 3

### Treatment of Individuals Refractory to Standard IV Diuretic Therapy

A double-blind, randomized, multi-site, placebo controlled study was conducted as follows: Approximately 35 subjects presenting with congestive heart failure who were factory to high dose diuretic therapy were randomized to KW-3902 IV:placebo in a 2:1 ratio in an ascending dose manner. Doses of 10 mg, 30 mg, and 60 mg KW-3902 IV or placebo were administered once over 120 minutes. All subjects had an estimated creatinine clearance between 20 mL/min and 80 mL/min. The mean baseline creatine clearance rate was 34.7 mL/min. Changes in urine output were measured hourly. The creatinine clearance rate was measured every three hours.

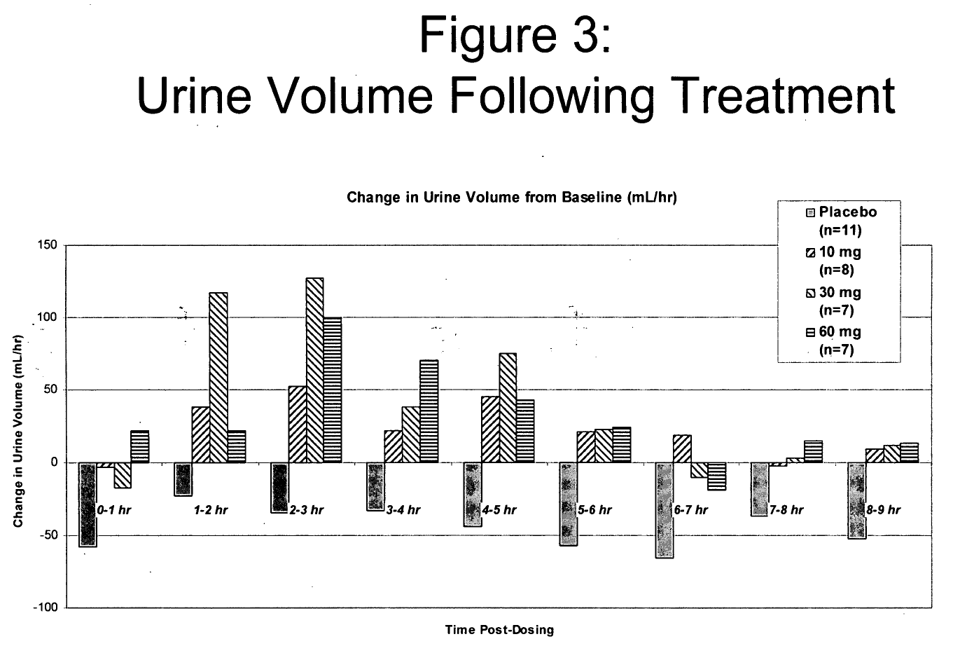

As shown in FIG. 3, all doses of KW-3902 resulted in increased hourly urine volume over the ensuing 9 hours compared to placebo, with the most meaningful increase taking place in individuals who received 30 mg KW-3902 over hours 1-2 and 203.

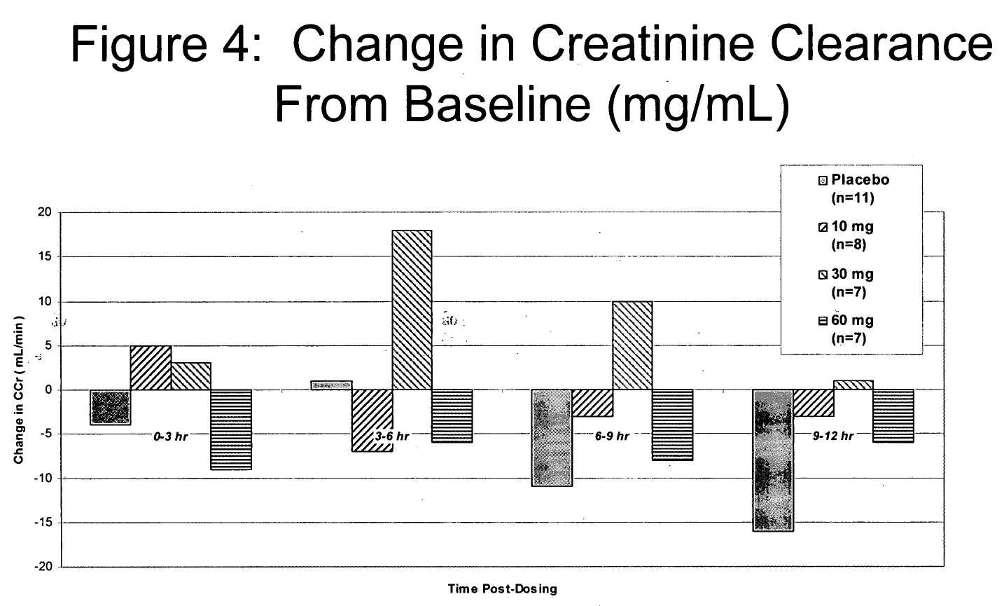

As shown in FIG. 4, administration of 10 mg KW-3902 resulted in an improvement in creatinine clearance over 0-3 hours. 30 mg KW-3902 also resulted in increased creatinine clearance rate over 0-12 hours. As discussed in Example 1, the beneficial effects on kidney function were not dose-dependent.

A hospitalized patient who has been treated with maximum amounts of IV diuretic and is still symptomatic, fluid overloaded, or whose urine output is less than fluid intake is evaluated for further treatment. A 10 mg dose of KW-3902 in injectable form is infused through the IV line. The patient receives continued treatment with furosemide, and also receives 10 mg of KW-3902 at 6 hour intervals, or more or less frequently as needed. The patient's fluid intake and output, urine volume, serum and urine creatinine levels, electrolytes and cardiac function are monitored.

At the discretion of the attending physician, the dosage of KW-3902 can be increased to 15 mg, 30 mg, 60 mg, or 100 mg either during the treatment or as the initial dose, or furosemide can be given as a continuous infusion.

### Example 4

### Treatment of Individuals Refractory to Standard IV Diuretic Therapy

A hospitalized patient who has been treated with maximum amounts of IV still symptomatic, fluid overloaded, or whose urine output is less than fluid ted for further treatment. A 10 mg dose of KW-3902 in injectable form is infused through the IV line. The patient receives continued treatment with furosemide, and also receives 10 mg of KW-3902 at 6 hour intervals, or more or less frequently as needed. The patient's fluid intake and output, urine volume, serum and urine creatinine levels, electrolytes and cardiac function are monitored.

At the discretion of the attending physician, the dosage of KW-3902 can be increased to 15 mg, 3Q mg, 60 mg, or 100 mg either during the treatment or as the initial dose, or furosemide can be given as a continuous infusion.

### Example 5

### Treatment of Individuals with Fluid Overload

A patient with fluid overload, as manifested by peripheral edema, dyspnea, and/or other signs or symptoms presents to the hospital, clinic, or doctor's office. In addition to standard of care therapy which would include IV diuretics, e.g., IV furosemide, bumetanide and/or oral metolazone, the patient is also given 2.5 mg of KW-3902 in injectable form. The patient is administered 2.5 mg of KW-3902 and 40 mg of furosemide at 24 hour intervals, or furosemide can be given as a continuous infusion. The patient's fluid intake and output, urine volume, serum and urine creatinine levels, electrolytes and cardiac function are monitored.

At the discretion of the attending physician, the dosage of KW-3902 can be increased to 15 mg, 30 mg, or 60 mg either during the treatment or as the initial dose. In addition, the dosage of furosemide can be increased to 60 mg, 80 mg, 100 mg, 120 mg, 140 mg, or 160 mg either during the treatment or as the initial dose. This treatment can be used for patients whether or not they suffer from renal impairment.

### Example 6

### Treatment of Individuals with Fluid Overload and Impaired Renal Function

A patient with fluid overload, as manifested by peripheral edema, dyspnea,.and/or other signs or symptoms presents himself to the physician's office or clinic. The patient has been on a therapy regimen that includes oral diuretics and, in addition, to needing a higher dose of diuretics to manage his/her fluid balance, the patient is now showing impaired renal function. The patient is prescribed 5 mg of KW-3902 to be taken orally, once daily, concurrent with other diuretic therapy. The patient's fluid intake and output, urine volume, serum and urine creatinine levels, electrolytes and cardiac function are monitored.

At the discretion of the attending physician, the dosage of KW-3902 can be increased to 15 mg, 30 mg, 60 mg, 80. mg, or 100 mg oral either during the treatment or as the initial dose. In addition, the dosage of furosemide can be increased to 60 mg, 80 mg, 100 mg, 120 mg, 140 mg, or 160 mg either during the treatment or as the initial dose.

### Example 7

### Treatment of Individuals with Fluid Overload

A patient with fluid overload, as manifested by peripheral edema, dyspnea, and/or other signs or symptoms presents to the physician's office or clinic. The patient has been on a therapy regimen that includes oral diuretics and needs a higher dose of diuretics to manage his/her fluid balance. To delay or prevent the onset of renal impairment and/or to delay the need to use higher dosages of standard diuretics, the patient is prescribed 5 mg of KW-3902 to be taken orally, once daily, concurrent with their diuretic therapy. The patient's fluid intake and output, urine volume, serum and urine creatinine levels, electrolytes and cardiac function are monitored.

At the discretion of the attending physician, the dosage of KW-3902 can be increased to 15 mg, 30 mg, 60 mg, 80 mg,.or 100 mg oral either during the treatment or as the initial dose. In addition, the dosage of furosemide can be increased to 60 mg, 80 mg, 100 mg, 120 mg, 140 mg, or 160 mg either during the treatment or as the initial dose.

### Example 8

### Treatment of Individuals with Congestive Heart Failure

A patient with congestive heart failure presents to the physician's office or clinic. The patient is put on a therapy regimen that includes oral diuretics to manage his/her fluid balance. To delay or prevent the onset of renal impairment and/or to delay the need to use higher dosages of standard diuretics, the patient is also prescribed 5 mg of KW-3902 to be taken orally, once daily, concurrent with their diuretic therapy. The patient's fluid levels, urine volume, serum and urine creatinine levels, electrolytes and cardiac function are monitored.

At the discretion of the attending physician, the dosage of KW-3902 can be increased to 15 mg, 30 mg, 60 mg, 80 mg, or 100 mg oral either during the treatment or as the initial dose. In addition, the dosage of furosemide can be increased to 60 mg, 80 mg, 100 mg, 120 mg, 140 mg, or 160 mg either during the treatment or as the initial dose.

### Example 9

### Improving Health Outcomes for of Individuals with Congestive Heart Failure

A patient with congestive heart failure presents to the physician's office or clinic. The patient is put on a therapy regimen that includes oral diuretics to manage his/her fluid balance. To improve overall health outcomes (i.e., morbidity or mortality rates due to CHF), the patient is also prescribed 5 mg of KW-3902 to be taken orally, once daily, concurrent with their diuretic therapy, or similar doses of KW-3902 is administered to the patient intravenously. The patient's fluid levels, urine volume, serum and urine creatinine levels, electrolytes and cardiac function are monitored.

At the discretion of the attending physician, the dosage of KW-3902 can be increased to 15 mg, 30 mg, 60 mg, 80 mg, or 100 mg either during the treatment or as the initial dose. In addition, the dosage of furosemide can be increased to 60 mg, 80 mg, 100 mg, 120 mg, 140 mg, or 160 mg either during the treatment or as the initial dose.

What is claimed is:

1. A method of treating an individual with impaired renal function, comprising
identifying an individual with impaired renal function
administering to said individual a pharmaceutical composition comprising less than or equal to about 10 mg KW-3902, or a pharmaceutically acceptable salt, ester, amide, metabolite, or prodrug thereof.

2. The method of claim 1, wherein said identification step comprises identifying an individual with a creatinine clearance rate of less than about 80 mg/dL to about 20 mg/dL.

3. The method of claim 1, wherein said individual suffers from congestive heart failure.

4. The method of claim 1, wherein said individual is not refractory to standard diuretic therapy.

5. The method of claim 1, wherein said individual is refractory to standard diuretic therapy.

6. The method of claim 1, wherein said pharmaceutical composition comprises about 2.5 mg KW-3902.

7. The method of claim 1, further comprising administering to said individual a therapeutically effective amount of a non-adenosine modifying diuretic.

8. The method of claim 7, wherein said non-adenosine modifying diuretic is a proximal diuretic.

9. The method of claim 7, wherein said non-adenosine modifying diuretic is a loop diuretic.

10. The method of claim 7, wherein said non-adenosine modifying diuretic is a distal diuretic.

11. The method of claim 7, wherein the diuretic is selected from the group consisting of hydrochlorothiazides, furosemide, torsemide, bumetanide, ethacrynic acid, piretanide, spironolactone, triamterene, and amiloridehiazides.

12. The method of claim 11, wherein the diuretic is furosemide.

13. The method of claim 7, wherein said KW-3902 and said non-adenosine modifying diuretic are administered substantially simultaneously.

14. The method of claim 6, wherein said KW-3902 and said non-adenosine modifying diuretic are administered sequentially.
